(19) United States
(12) Patent Application Publication
(10) Pub. No.: US 2018/0036526 A1
(43) Pub. Date: Feb. 8, 2018

(54) ELECTRICAL STIMULATION DEVICE

(71) Applicant: Y-BRAIN INC., Daejeon (KR)

(72) Inventors: Ki Won LEE, Daejeon (KR); Cheon Ju KO, Yongin-si (KR); Jong Min JANG, Suwon-si (KR); Byung Gik KIM, Daegu (KR)

(73) Assignee: Y-BRAIN INC., Daejeon (KR)

(21) Appl. No.: 15/789,218

(22) Filed: Oct. 20, 2017

Related U.S. Application Data
(63) Continuation of application No. PCT/KR2016/004287, filed on Apr. 25, 2016.

(30) Foreign Application Priority Data
Apr. 27, 2015 (KR) 10-2015-0058791

(51) Int. Cl. A61N 1/04; A61N 1/36

(52) U.S. Cl. CPC A61N 1/0492; A61N 1/36028

(57) ABSTRACT

Disclosed is an electrical stimulation device. The electrical stimulation device includes at least one electrode unit that contacts the head of a user to apply a current to the head of the user, and a current providing module that provides a current to the at least one electrode unit, wherein the current providing module includes a plurality of pads disposed on a path for providing the current to the at least one electrode unit to be spaced apart from each other, and wherein the current providing module provides the current to the at least one electrode unit if the plurality of pads are connected to each other by a conductive material.

### CROSS-REFERENCE TO RELATED APPLICATIONS

The present application is a continuation of International Patent Application No. PCT/KR2016/004287, filed on Apr. 25, 2016, which is based upon and claims the benefit of priority to Korean Patent Application No. 10-2015-0058791, filed on Apr. 27, 2015. The disclosures of the above-listed applications are hereby incorporated by reference herein in their entirety.

### BACKGROUND

Embodiments of the inventive concept described herein relate to an electrical stimulation device, and more particularly, relate to an electrical stimulation device for applying an electrical stimulus to the head of a user.

Meanwhile, a brain electrical stimulation technology using transcranial direct current stimulation (tDCS) is known to be effective to improve recognition ability and treat mental diseases such as depressions or attention deficit hyperactivity disorders (ADHD).

Accordingly, if the brain electrical stimulation technology may be used in everyday lives, the brain function may be improved, and mental diseases may be continuously treated by activating or retaining connections between nerves.

A tDCS device according to the related art includes one or more electrodes for applying electrical stimuli, and each of the electrodes includes a patch layer that contacts skin of the head of the user and an electrode layer that transfers a current to the patch layer. The patch layer contains an electrolyte for flows of currents, and thus an oxidation/reduction reaction may occur on an interface between the patch layer and the electrode layer. The oxidation/reduction reaction gradually acidifies or basifies the patch layer, and if the pH index of the patch layer deviates from a threshold range due to the repeated use of the tDCS device, the skin of the head of the user, which contacts the patch layer, may be burned.

### SUMMARY

Embodiments of the inventive concept provide a disposable electrical stimulation device that may prevent skin of the head of the user from being burned or damaged when the user uses the electrical stimulation device.

In accordance with an aspect of the inventive concept, there is provided an electrical stimulation device including at least one electrode unit that contacts the head of a user to apply a current to the head of the user, and a current providing module that provides a current to the at least one electrode unit, wherein the current providing module includes a plurality of pads disposed on a path for providing the current to the at least one electrode unit to be spaced apart from each other, and wherein the current providing module provides the current to the at least one electrode unit if the plurality of pads are connected to each other by a conductive material.

In some embodiments, the electrical stimulation device may further include a flexible substrate on which the current providing module is disposed.

In some embodiments, the conductive material may be at least one of conductive silicon, a conductive tape, a conductive wire, a conductive film, conductive paste, and a part of a human body.

In some embodiments, the conductive material may be disposed on the plurality of pads, the current providing module may further include a nonconductive material disposed between the plurality of pads and the conductive material, and the plurality of pads may be connected to each other by the conductive material if the nonconductive material is removed between the plurality of pads and the conductive material.

Further, the nonconductive material may be at least one of an insulation sheet, an insulation tape, an insulation paper, or an insulation film.

In some embodiments, the current providing module may not provide the current to the at least one electrode unit if a predetermined reference use time period elapses after the current is provided to the at least one electrode unit.

Further, the current providing module may further include a power supply unit that supplies a power source if the plurality of pads are connected to each other by the conductive material, and a current providing unit that provides a current of a predetermined value to the at least one electrode unit by using the supplied power source, and the current providing unit may be disabled if the predetermined reference use time period elapses after the current is provided to the at least one electrode unit.

Further, the current providing module may further include a control unit that controls an operation of the current providing module, and the control unit may enable an operation of the current providing unit if the plurality of pads are connected to each other by the conductive material, and disenables an operation of the current providing unit if the predetermined reference use time period elapses after the current is provided to the at least one electrode unit.

Meanwhile, the current providing module may further include a control unit that controls an operation of the current providing module, the control unit may include a memory that initially stores a use state of the electrical stimulation device as an unused state, and updates the use state of the electrical stimulation device to a used state and stores the updated use state of the electrical stimulation device if a predetermined reference use time period elapses after the current is provided to the at least one electrode unit, and the control unit may disable an operation of the current providing module if the use state of the electrical stimulation device is a used state.

Further, the current providing module may further include a power supply unit that supplies a power source if the plurality of pads are connected to each other by the conductive material, and a current providing unit that provides a current of a predetermined value to the at least one electrode unit by using the supplied power source, and the control unit may enable an operation of the current providing unit if the use state of the electrical stimulation device is an unused state, and may disable an operation of the current providing unit if the use state of the electrical stimulation device is a used state.

In some embodiments, the current providing module may include a control unit that controls an operation of the current providing module, the current providing module may supply a power source to the control unit if the plurality of pads are connected to each other by the conductive material, and the control unit may control the current providing module to provide the current to the at least one electrode unit.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a block diagram illustrating a schematic configuration of an electrical stimulation device according to an embodiment of the inventive concept;

FIG. 2 is a view illustrating that a plurality of pads of the electrical stimulation device of FIG. 1 are connected to each other by a conductive material;

FIG. 3 is a view illustrating a schematic configuration of a current providing module of FIG. 1;

FIG. 4 is a view illustrating an example of using the electrical stimulation device of FIG. 1; and

FIG. 5 is a view illustrating a schematic configuration and a schematic operation of an electrode unit of the electrical stimulation device of FIG. 1.

### DETAILED DESCRIPTION

The above and other aspects, features and advantages of the invention will become apparent from the following description of the following embodiments given in conjunction with the accompanying drawings. However, the inventive concept is not limited to the embodiments disclosed below, but may be implemented in various forms. The embodiments of the inventive concept are provided to make the disclosure of the inventive concept complete and fully inform those skilled in the art to which the inventive concept pertains of the scope of the inventive concept.

The terms used herein are provided to describe the embodiments but not to limit the inventive concept. In the specification, the singular forms include plural forms unless particularly mentioned. The terms “comprises” and/or “comprising” used herein does not exclude presence or addition of one or more other elements, in addition to the aforementioned elements. Throughout the specification, the same reference numerals dente the same elements, and “and/or” includes the respective elements and all combinations of the elements. Although “first”, “second” and the like are used to describe various elements, the elements are not limited by the terms. The terms are used simply to distinguish one element from other elements. Accordingly, it is apparent that a first element mentioned in the following may be a second element without departing from the spirit of the inventive concept.

Unless otherwise defined, all terms (including technical and scientific terms) used herein have the same meaning as commonly understood by those skilled in the art to which the inventive concept pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

Hereinafter, exemplary embodiments of the inventive concept will be described in detail with reference to the accompanying drawings.

“Electrical stimulation” that will be mentioned in the following may refer to transcranial current stimulation (tCS) such as transcranial direct current stimulation (tDCS), transcranial alternating current stimulation (tACS), and transcranial random-noise stimulation, but the inventive concept is not limited thereto.

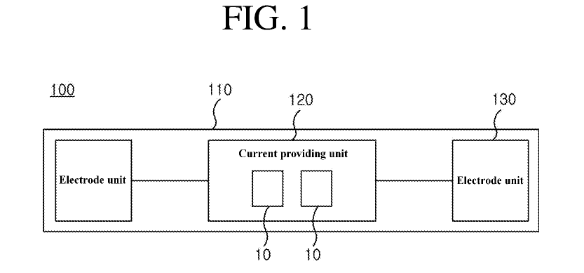

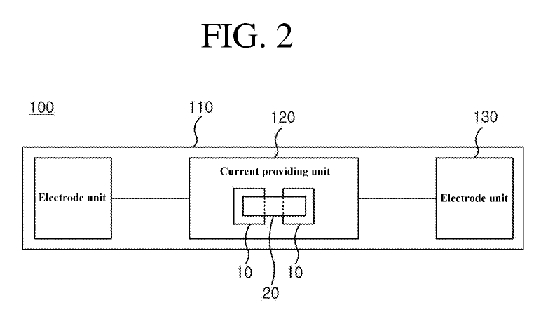

FIG. 1 is a block diagram illustrating a schematic configuration of an electrical stimulation device according to an embodiment of the inventive concept. FIG. 2 is a view illustrating that a plurality of pads of the electrical stimulation device of FIG. 1 are connected to each other by a conductive material.

Referring to FIG. 1, the electrical stimulation device 100 according to the embodiment of the inventive concept includes a substrate 110, a current providing module 120 disposed on the substrate 110, and an electrode unit 130.

The substrate 110 may be a printed circuit board (PCB). A circuit wiring line for the current providing module 120 may be formed on a surface or in the interior of the substrate 110. The substrate 110 may be flexible.

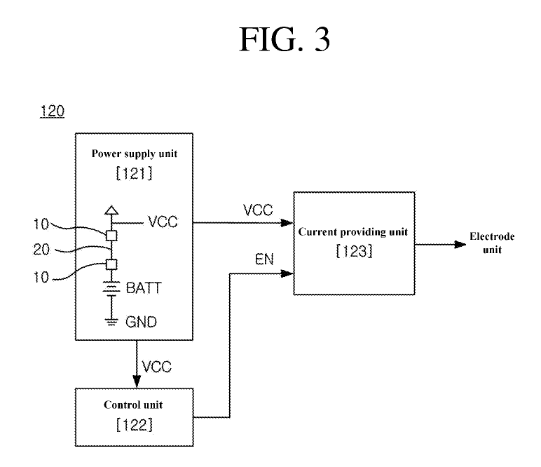

The current providing module 120 provides a current to the electrode unit 130. The current providing module 120 includes a plurality of pads 10 that are spaced apart from each other. The plurality of pads 10 are disposed on a path along which the current providing module 120 provides a current to the electrode unit 130. Because the plurality of pads 10 are not electrically connected to each other in the state of FIG. 1, the plurality of pads 10, the current providing module 120 cannot provide a current to the electrode unit 130 (that is, a disable state). Further, as illustrated in FIG. 2, after the plurality of pads 10 are electrically connected to each other by the conductive material 20, the current providing module 120 may provide a current to the electrode unit 130 (that is, an enable state). Detailed configuration and operation of the current providing module 120 will be described below in more detail with reference to FIG. 3.

For example, the conductive material 20 may be one or a combination of conductive silicon, a conductive tape, a conductive wire, a conductive film, and conductive paste, or a material that is well known in the art to which the inventive concept pertains. Further, the conductive material 20 may be a part (skin of the head or the like) of a human body.

In some embodiments, through an operation (for example, a mechanical switching operation) of the user, the conductive material 20 contacts the plurality of pads 10 to electrically connect the plurality of pads 10. The conductive material 20 may contact only one of the plurality of pads 10, and may contact the remaining ones of the plurality of pads 10 through an operation of the user. Alternatively, the conductive material 20 may contact none of the plurality of pads 10, and may contact all of the plurality of pads 10 through an operation of the user.

In some embodiments, the conductive material 20 may be disposed on the plurality of pads 10, and the nonconductive material (not illustrated) may be interposed between the conductive material 20 and the plurality of pads 10. Due to the nonconductive material 20, the conductive material 20 may be prevented from contacting the plurality of pads 10, thereby failing to electrically connect the plurality of pads 10. Further, if the nonconductive material 20 is removed between the conductive material 20 and the plurality of pads 10 through an arbitrary operation of the user, the conductive material 20 may contact the plurality of pads 10 to electrically connect the plurality of pads 10. The nonconductive material 20 may be interposed between the conductive material 20 and one of the plurality of pads 10, or may be interposed between the conductive material 20 and all of the plurality of pads 10. The nonconductive material 20 may be provided in the form of an insulation sheet, an insulation tape, an insulation paper, or an insulation film, but the inventive concept is not limited thereto.

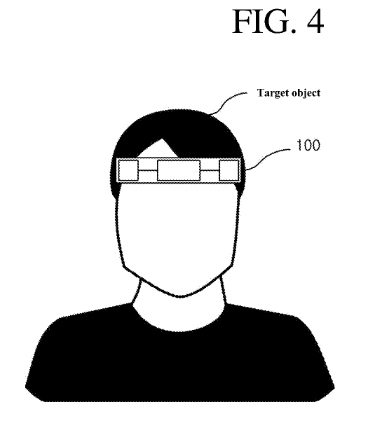

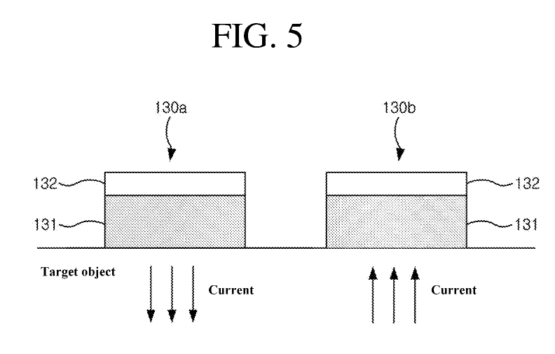

The electrode unit 130 contacts the head of the user (a target object) and applies a current to the head of the user. The user may mount the electrical stimulation device 100 on the head or may attach the electrical stimulation device 100 to the head, and thus the electrode unit 130 may contact the head of the user. Detailed configuration and operation of the electrode unit 130 will be described below in more detail with reference to FIGS. 4 and 5.

Meanwhile, the elements of FIG. 1 are not essential to the electrical stimulation device 100 according to the embodiment of the inventive concept, and thus the electrical stimulation device 100 may include more or less elements.

Further, the overall shape of the electrical stimulation device 100 is not limited to the embodiment of FIG. 1. Unlike FIG. 1, at least a part of the electrode unit 130 may not be disposed on the substrate 110. Further, the wiring line that connects the current providing module 120 and the electrode unit 130 may be configured to be serpentine such that the distances between the plurality of electrode units 130 may be adjusted (increased).

Hereinafter, detailed configuration and operation of the current providing module will be described with reference to FIG. 3. FIG. 3 is a view illustrating a schematic configuration of a current providing module of FIG. 1.

Referring to FIG. 3, the current providing module 120 includes a power supply unit 121, a control unit 122, and a current providing unit 123.

The power supply unit 121 supplies a power source VCC of a first voltage (for example, 3.3 V) to the control unit 122 and the current providing unit 123 as well as a battery. For example, the battery may be a single-use battery. As illustrated in FIG. 3, the plurality of pads 10 may be disposed in a path between a power supply terminal of the power supply unit 121 and the battery. If the plurality of pads 10 are electrically connected to each other by the conductive material 20, the power supply terminal and the battery may be electrically connected to each other so that the power supply unit 121 may supply a power source VCC.

The control unit 122 generally controls an operation of the current providing module 120. If a power source VCC is supplied from the power supply unit 121, the control unit 122 may be operated. A natural or arbitrary time delay may exist between the supply of power and an operation of the control unit 122. The control unit 122 may transmit a control signal EN to the current providing unit 123 to control an operation of the current providing unit 123.

The control unit 122 may include a memory (not illustrated) (for example, a register) for storing a use state of the electrical stimulation device 100. The use state of the electrical stimulation device 100 may be classified into an unused state and a used state. Initially, the memory of the control unit 122 may store the use state of the electrical stimulation device 100 as an unused state. If determining that the electrical stimulation device 100 has completely used a reference number of times (for example, one time), the control unit 122 may store the use state of the electrical stimulation device 100 in the memory as a used state. The control unit 122 may count a lapse time period after the power source VCC is applied, and if the predetermined reference use time period elapses, may determine that the electrical stimulation device 100 has completely used the reference number of times.

The control unit 122 may transmit another control signal EN to the current providing unit 123 according to the use state of the electrical stimulation device 100. If the use state of the electrical stimulation device 100 is an unused state, the control unit 122 may enable an operation of the current providing unit 123 by transmitting a control signal EN of a first level (for example, HIGH). Accordingly, the current providing unit 123 provides a current to the electrode unit 130. If the use state of the electrical stimulation device 100 is a used state, the control unit 122 may disable an operation of the current providing unit 123 by transmitting a control signal EN of a second level (for example, LOW). Accordingly, the current providing unit 123 does not provide a current to the electrode unit 130.

For example, the control unit 122 may include a processor, a microprocessor, a micro controller, a central processing unit (CPU), a micro processing unit (MPU), and a micro controller unit (MCU). The control unit 122 may include firmware for performing the above-mentioned control operation.

The current providing unit 123 provides a current to the electrode unit 130. If a power source VCC is supplied from the power supply unit 121 and the control unit 122 transmits a control signal EN of a first level, the current providing unit 123 may be operated. The current providing unit 123 may boost a first voltage supplied from the power supply unit 121 to a second voltage (for example, 10 V), and may provide a current of a predetermined value (for example, 1 mA) to the electrode unit 130 by using the boosted second voltage. To achieve this, the current providing unit 123 may include a voltage booster circuit and a static current circuit (for example, a current regulator diode (CRD)).

Hereinafter, detailed configuration and operation of the electrode unit will be described with reference to FIGS. 4 and 5. FIG. 4 is a view illustrating an example of using the electrical stimulation device of FIG. 1. FIG. 5 is a view illustrating a schematic configuration and a schematic operation of an electrode unit of the electrical stimulation device of FIG. 1.

Referring to FIG. 4, the user (target object) may attach the electrical stimulation device 100 to the head of the user. Unlike FIG. 4, the user may mount the electrical stimulation device 100 on the head of the user. As the user attaches the electrical stimulation device 100 to the head, the electrode unit 130 may contact the head of the user. The electrode unit 130 may contact the forehead of the user, but the inventive concept is not limited thereto.

As described below, a plurality of electrode units 130 (a first electrode 130a and a second electrode 130b) may be provided. By using the plurality of electrode units 130, the electrical stimulation device 100 may apply a current to a plurality of parts (locations or areas) of the head of the user. For example, one of the electrode units 130 may contact the left side of the head of the user, and the other of the electrode units 130 may contact the right side of the head of the user. According to an embodiment, a plurality of electrode units 130 may be controlled to be operated independently.

Referring to FIG. 5, each of the electrodes 130 includes a patch layer 131 and an electrode layer 132.

The patch layer 131 is a layer that directly contacts skin of the head of the user. The patch layer 131 may include a single layer. The patch layer 131 applies a current transferred from the electrode layer 132 to the head of the user. To achieve this, the patch layer 131 may include an electrolyte for transferring a current. For example, the patch layer 131 may include sponge or hydrogel that contains an electrolyte, but the inventive concept is not limited thereto. The electrolyte may include chlorine ions (Cl₋) that are commonly present in skin of the user. The patch layer 131 may be formed of a material having a relatively high impedance.

The electrode layer 132 is formed on the patch layer 131. The electrode layer 132 does not contact skin of the head of the user. The electrode layer 132 delivers a current to the patch layer 131 to apply an electrical stimulus to the user through the patch layer 131. For example, the electrode layer 132 may include a conductive carbon sheet or conductive silicon, but the inventive concept is not limited thereto.

In some embodiments, each of the electrode units 130 may include a plurality of segments, and the plurality of segments may be formed on the substrate to be spaced apart from each other. Each of the segments may include the patch layer 131 and the electrode layer 132. The number, shapes, intervals of the segments may be variously modified according to embodiments. The plurality of segments may be controlled independently. Further, several segments may constitute a group to be controlled.

During an operation of the electrical stimulation device 100, the first electrode unit 130a of the plurality of electrode units 130 may function as an anode and the second electrode unit 130b may function as a cathode. Further, an oxidation/reduction reaction (a water splitting reaction) occurs on an interface between the patch layer 131 and the electrode layer 132 when a current is transferred from the electrode layer 132 to the patch layer 131. The patch layer 131 may include water (used as a solvent) in addition to the electrolyte, and the water of the patch layer 131 reacts electrons provided by the electrode layer 132 to be split into hydroxide ions and hydrogen molecules (in the case of a cathode). Further, the water of the patch layer 131 may lose electrons to be split into hydrogen ions (H+) and oxygen molecules (in the case of an anode). Accordingly, in the case of a cathode electrode unit 130b, the pH index of the patch layer 131 may be gradually increased by hydroxyl ions, and similarly, in the case of an anode electrode unit 130a, the pH index of the patch layer 131 may be gradually decreased by hydrogen ions. Further, if the change of the pH index of the patch layer 100 deviates from a threshold range due to the repeated use of the patch layer 131, the skin of the head of the user, which contacts the patch layer 131, may be burned.

However, according to the electrical stimulation device 100 according to the embodiments of the inventive concept, because the user cannot repeat the electrical stimulation device 100, the electrodes of the electrical stimulation device 100 may be prevented from being acidized or basified by an oxidation/reduction reaction. Further, according to the electrical stimulation device 100 according to the embodiments of the inventive concept, because the pH indexes of the electrodes of the electrical stimulation device 100 may be maintained in a safe range, skin of the head of the user, which contacts the patch layer 131, may be prevented from being burned or damaged.

According to the disclosed embodiment, by providing a disposable electrical stimulation device, a user may repeatedly use the electrical stimulation device and electrodes of the electrical stimulus device may be prevented from being acidified or basified by an oxidation/reduction reaction.

Further, According to the disclosed embodiment, because the pH indexes of the electrodes of the disposable electrical stimulation device are maintained in a safe range, skin of the head of the user, which contacts a patch layer, may be prevented from being burned or damaged.

Although the embodiments of the electrical stimulation devices 100 that apply an electrical stimulus to the head of the user have been described in the specification, the technical features of the inventive concept also may be applied to the electrical stimulation device for applying an electrical stimulus to another body portion of the user in addition to the head of the user in substantially the same manner.

Although the exemplary embodiments of the inventive concept have been described with reference to the accompanying drawings, it will be understood by those skilled in the art to which the inventive concept pertains that the inventive concept can be carried out in other detailed forms without changing the technical spirits and essential features thereof. Therefore, the above-described embodiments are exemplary in all aspects, and should be construed not to be restrictive.

What is claimed is:

1. An electrical stimulation device comprising:
at least one electrode unit that contacts the head of a user to apply a current to the head of the user; and
a current providing module that provides a current to the at least one electrode unit,
wherein the current providing module includes a plurality of pads disposed on a path for providing the current to the at least one electrode unit to be spaced apart from each other, and
wherein the current providing module provides the current to the at least one electrode unit if the plurality of pads are connected to each other by a conductive material.

2. The electrical stimulation device of claim 1, further comprising:
a flexible substrate on which the current providing module is disposed.

3. The electrical stimulation device of claim 1, wherein the conductive material is at least one of conductive silicon, a conductive tape, a conductive wire, a conductive film, conductive paste, and a part of a human body.

4. The electrical stimulation device of claim 1, wherein the conductive material is disposed on the plurality of pads,
wherein the current providing module further includes a nonconductive material disposed between the plurality of pads and the conductive material, and
wherein the plurality of pads are connected to each other by the conductive material if the nonconductive material is removed between the plurality of pads and the conductive material.

5. The electrical stimulation device of claim 4, wherein the nonconductive material is at least one of an insulation sheet, an insulation tape, an insulation paper, or an insulation film.

6. The electrical stimulation device of claim 1, wherein the current providing module does not provide the current to the at least one electrode unit if a predetermined reference use time period elapses after the current is provided to the at least one electrode unit.

7. The electrical stimulation device of claim 6, wherein the current providing module further includes:
a power supply unit that supplies a power source if the plurality of pads are connected to each other by the conductive material; and
a current providing unit that provides a current of a predetermined value to the at least one electrode unit by using the supplied power source, and
wherein the current providing unit is disabled if the predetermined reference use time period elapses after the current is provided to the at least one electrode unit.

8. The electrical stimulation device of claim 7, wherein the current providing module further includes a control unit that controls an operation of the current providing module, and
wherein the control unit enables an operation of the current providing unit if the plurality of pads are connected to each other by the conductive material, and disenables an operation of the current providing unit if the predetermined reference use time period elapses after the current is provided to the at least one electrode unit.

9. The electrical stimulation device of claim 6, wherein the current providing module further includes:
a control unit that controls an operation of the current providing module,
wherein the control unit includes a memory that initially stores a use state of the electrical stimulation device as an unused state, and updates the use state of the electrical stimulation device to a used state and stores the updated use state of the electrical stimulation device if a predetermined reference use time period elapses after the current is provided to the at least one electrode unit, and
wherein the control unit disables an operation of the current providing module if the use state of the electrical stimulation device is a used state.

10. The electrical stimulation device of claim 9, wherein the current providing module further includes:
a power supply unit that supplies a power source if the plurality of pads are connected to each other by the conductive material; and
a current providing unit that provides a current of a predetermined value to the at least one electrode unit by using the supplied power source, and
wherein the control unit enables an operation of the current providing unit if the use state of the electrical stimulation device is an unused state, and disables an operation of the current providing unit if the use state of the electrical stimulation device is a used state.

11. The electrical stimulation device of claim 1, wherein the current providing module includes a control unit that controls an operation of the current providing module,
wherein the current providing module supplies a power source to the control unit if the plurality of pads are connected to each other by the conductive material, and
wherein the control unit controls the current providing module to provide the current to the at least one electrode unit.
